(19) United States
(12) Patent Application Publication
(10) Pub. No.: US 2014/0073766 A1
(43) Pub. Date: Mar. 13, 2014

(54) METHODS FOR PURIFYING IgY ANTIBODIES

(71) Applicant: AVIANAX LLC, Grand Forks, ND (US)

(72) Inventors: Thomas A. Henderson, Grand Forks, ND (US); Subramanian V. Iyer, Hockessin, DE (US); Jeremy Vrchota, Grand Forks, ND (US); James Schiltz, Sisseton, SD (US)

(21) Appl. No.: 14/020,469

(22) Filed: Sep. 6, 2013

Related U.S. Application Data
(60) Provisional application No. 61/698,274, filed on Sep. 7, 2012.

(51) Int. Cl. C07K 16/02

(52) U.S. Cl. CPC C07K 16/02
USPC 530/387.1

(57) ABSTRACT

A method for purifying IgY antibodies is provided. The method comprises precipitating IgY antibodies from a sample by contacting the sample with a precipitating agent; and differentially precipitating the IgY antibodies obtained in step (a) using the same or different precipitating agent to separate IgY(Fc) and IgY(ΔFc) antibodies.

### FIELD OF THE INVENTION

The present invention is directed to a method for purifying antibodies, particularly avian IgY antibodies, and the IgY antibodies obtained thereto. The method includes differential precipitation of IgY antibodies and further purification of precipitated antibodies, preferably using chromatographic techniques.

### BACKGROUND

Antibodies are used extensively for research, therapeutic and diagnostic purposes. Although monoclonal antibodies are popular for their diagnostic and therapeutic potential, polyclonal antibodies also have several therapeutic applications. For example, polyclonal antibodies can be administered as passive therapeutic agents to provide immunity against pathogenic agents for which effective vaccines are not yet available (U.S. Pat. No. 8,029,785). Polyclonal antibodies could also be used to provide immunity to individuals who have not been previously vaccinated at the time of an epidemic outbreak. Additionally, polyclonal antibodies are also useful for providing immunity against pathogens where the epitope is not conservatively maintained, such as a pathogen having numerous species or viral pathogens that are prone to a higher mutation frequency (U.S. Pat. No. 8,029,785).

In the field of in vitro immunodiagnostics, an intact antibody molecule is not preferred since the Fc portion of intact antibodies binds to Fc receptors on immune cells, activates the complement system or reacts with rheumatoid factors in mammalian sera causing interference in immunoassays. Therefore, for diagnostic purposes, antibodies that lack Fc portion are desired in order to avoid cross-reactions (Chiou. Duck antibodies for IVD applications, IVD Technology [online], [retrieved on 2012 Jun. 15]. Retrieved from the Internet <URL: http://www.ivdtechnology.com/article/naturally-existing-analog-mammalian-antibodies-offers-alternative-broad-applications>).

Polyclonal antibodies obtained from certain birds have shown promising therapeutic and diagnostic applications. Specifically, IgY antibodies obtained from duck and goose are useful as therapeutic agents and diagnostic reagents. For example, Applicant's own U.S. Pat. No. 8,029,785 to Schiltz et al. discloses methods for treating and preventing infection in mammals by administering polyclonal goose IgY antibodies obtained from infected or immunized geese. Ducks and geese produce two isoforms of IgY antibodies, a full length isoform (IgY(Fc)) and a truncated isoform (IgY(ΔFc)) (EP1371665B1). Avian IgY antibodies are functional equivalents of mammalian IgG antibodies and are also found in some reptiles and amphibians (Chiou. Duck antibodies for IVD applications, IVD Technology [online], [retrieved on 2012 Jun. 15]. Retrieved from the Internet <URL: http://www.ivdtechnology.com/article/naturally-existing-analog-mammalian-antibodies-offers-alternative-broad-applications>). Avian IgG antibodies are referred to as IgY antibodies because of their presence in egg yolk besides serum. The intact IgY(Fc) antibody possesses two heavy and two light chains, with the heavy chain having one variable and four constant domains. The molecular weight of IgY(Fc) is about 180 kDa and it has a sedimentation coefficient of 7.8 Svedberg units (S) (Chiou. Duck antibodies for IVD applications, IVD Technology [online], [retrieved on 2012 Jun. 15]. Retrieved from the Internet <URL: http://www.ivdtechnology.com/article/naturally-existing-analog-mammalian-antibodies-offers-alternative-broad-applications>).

The truncated form of IgY(Fc) lacks two Fc domains (C_{H}3 and C_{H}4) of the heavy chain and is similar to a F(ab′)₂ fragment of an IgG antibody. IgY(ΔFc) has a molecular weight of about 120 kDa and a sedimentation coefficient of 5.7 S (Chiou. Duck antibodies for IVD applications, IVD Technology [online], [retrieved on 2012 Jun. 15]. Retrieved from the Internet <URL: http://www.ivdtechnology.com/article/naturally-existing-analog-mammalian-antibodies-offers-alternative-broad-applications>). IgY(ΔFc) antibodies are present only in birds that belong to the order Anseriformes, such as duck and goose (EP1371665B1).

Egg yolk of goose or duck eggs contains a high amount of IgY antibodies and therefore serves as a good source of IgY antibodies. However, high lipid content of egg yolk makes the purification of IgY antibodies difficult (EP1371665B1). Many efforts directed to the isolation of IgY antibodies have been made. For example, Nguyen et al. (PLOS One, 2010, 5(4), e10152) discloses a method for isolating IgY antibodies from chicken egg yolk by precipitating IgY-containing yolk supernatant with ammonium sulfate at 60% concentration. The precipitated IgY antibodies were dialyzed and used for intranasal administration of mice.

Tong et al. (Biochemical Engineering Journal, 2011, 56(3), 205-211) discloses a method for purifying IgY(ΔFc) from goose plasma using hydrophobic charge induction chromatography (HCIC). Specifically, goose plasma was precipitated using caprylic acid and IgY(ΔFc) antibodies present in the supernatant were purified using HCIC.

EP1371665B1 discloses a process for selectively isolating IgY antibodies from egg yolk of an anseriform bird. The process of EP1371665B1 comprises (a) absorbing IgY antibodies present in yolk suspension onto a water insoluble non-charged active constituent in the stationary phase; (b) flowing the water insoluble non-charged absorbent with a buffer to obtain an aqueous fraction containing the yolk antibodies; (c) salting out the aqueous fraction containing yolk antibodies in step (b) with (NH₄)₂SO₄ at a first concentration ranging from about 15% (w/v) to about 24% (w/v); and (d) salting out the aqueous fraction containing yolk antibodies treated in step (c) with (NH₄)₂SO₄ at a second concentration ranging from about 25% (w/v) to about 40% (w/v).

However, there still exists a need for more effective method for purifying the two isoforms of IgY antibodies.

### SUMMARY OF THE INVENTION

The present invention provides a simple, rapid and economic method for selectively purifying IgY(Fc) and IgY(ΔFc) antibodies. The IgY antibodies obtained using the methods of the invention can be used to provide protection against pathological agents or for immunodiagnostic purposes. Preferably, the IgY(Fc) antibodies obtained using the methods of the invention can be administered to provide protection against pathological agents, and the IgY(ΔFc) antibodies obtained using the methods of the invention can be used for immunodiagnostic purposes.

The method for purifying IgY antibodies according to the invention comprises (a) precipitating IgY antibodies from a sample by contacting the sample with a precipitating agent; and (b) differentially precipitating the IgY antibodies obtained in step (a) using the same or different precipitating agent to separate IgY(Fc) and IgY(ΔFc) antibodies. The method for purifying IgY antibodies may further comprise a step of purifying one or both of the IgY(Fc) and IgY(ΔFc) antibodies obtained in step (b).

In one aspect of the invention, the method for purifying IgY antibodies comprises:
(a) contacting a suspension containing IgY antibodies with a precipitating agent at a first concentration to obtain a first precipitate containing the IgY antibodies;
(b) contacting the precipitate containing the IgY antibodies obtained in step (a) with the same or different precipitating agent at a second concentration to obtain a second precipitate containing a majority of one IgY isoform and a supernatant containing a majority of the other IgY isoform, wherein the second concentration is less than the first concentration;
(c) contacting the supernatant obtained in step (b) with the same or different precipitating agent at a third concentration to obtain a third precipitate containing a majority of the other IgY isoform, wherein the third concentration is more than the second concentration; and
(d) purifying the antibodies obtained in steps (b) and/or (c).

The order in which the two isoforms are precipitated from the sample may vary depending on the nature of the precipitating agent and the conditions under which precipitation is conducted. Although in a preferred embodiment, the second concentration is less that the first concentration and the third concentration is more than the second concentration, it is also within the scope of the invention that the second concentration is more than the first concentration and the third concentration is less than the second concentration.

In one aspect of the invention, the precipitated IgY(Fc) and IgY(ΔFc) antibodies are further purified, preferably using chromatographic techniques. In a preferred embodiment, hydrophobic charge induced chromatography (HCIC) is used to purify the precipitated isoforms of IgY antibodies. Purification of precipitated IgY antibodies using other chromatographic techniques such as adsorption chromatography, ion-exchange chromatography, size exclusion chromatography or affinity chromatography are also within the scope of the invention. Non-chromatographic purification techniques include differential centrifugation or affinity purification.

The present invention provides a simple, rapid and economic method for obtaining high yields of purified IgY(Fc) and IgY(ΔFc) antibodies. Preferably, the purity of IgY(Fc) antibodies obtained using the method of invention is at least 70% and more preferably, at least 90% and purity of IgY(ΔFc) antibodies obtained using the method of invention is at least 70%, and more preferably, at least 90%.

The present invention also relates to isolated IgY(Fc) and isolated IgY(ΔFc) antibodies. In a preferred embodiment, isolated IgY(Fc) and isolated IgY(ΔFc) antibodies are directed against an infectious agent. In one aspect, isolated IgY(Fc) and isolated IgY(ΔFc) antibodies against an infectious agent are isolated from goose eggs according to the methods of the invention.

The present invention also relates to pharmaceutical compositions comprising the purified IgY antibodies against a transmittable viral disease obtained by the method of the invention and to the use of IgY antibodies obtained by the method of the invention. In one embodiment, pharmaceutical compositions comprising the purified IgY(Fc) and/or IgY(ΔFc) antibodies obtained using the method of the invention can be administered to mammals or birds for treating or preventing infection. In particular embodiments, the compositions of the invention are useful for reducing mortality in animals that become infected or are infected with a transmittable viral disease. In one aspect, compositions comprising the purified IgY antibodies obtained by the method of the invention can be administered for treatment or prophylaxis of a transmittable viral disease in a subject. The subject can be a mammalian subject or an avian subject. Preferably, the compositions comprising the purified IgY antibodies obtained according to the present invention are of high neutralization titer. The compositions comprising IgY antibodies against an infectious agent may further include suitable salts, excipients and/or stabilizing agents.

In one aspect, the present invention provides a method for treatment or prophylaxis of a transmittable viral disease in a subject. In certain embodiments, the method comprises administering to the subject an amount of a composition effective for treating or preventing the infection, wherein the composition comprises IgY antibodies against the transmittable viral disease. In one particularly preferred embodiment, the IgY antibodies are goose IgY antibodies. Particularly, the treatment or prophylaxis can be evidenced by a reduction in mortality in a population affected by, or subject to infection by, the transmittable viral disease.

Preferably, IgY(ΔFc) antibodies purified according to the method of the invention does not activate the complement system or does not bind to rheumatoid factors of mammalian sera. Accordingly, IgY(ΔFc) antibodies obtained according to the present invention can be used for clinical and research purposes.

### BRIEF DESCRIPTION OF THE DRAWINGS

FIG. 1 shows a coomassie stained SDS-PAGE gel of precipitates obtained during ammonium sulfate precipitation. Lane 1: molecular weight standards; lane 2: yolk supernatant; lane 3: precipitate obtained after the first ammonium sulfate precipitation; lane 4: precipitate obtained after the second ammonium sulfate precipitation; lane 5: precipitate obtained after the third ammonium sulfate precipitation.

FIG. 2 shows a coomassie stained 4-15% gradient SDS-PAGE gel of fractions obtained during HCIC purification of IgY(Fc) antibodies. Lane 1: molecular weight standards; lane 2: 1:5 dilution of second ammonium sulfate precipitate; lane 3: flow through fraction #7 collected during washing of the FPLC column with Buffer A; lane 4: flow through fraction #8 collected during washing of the FPLC column with Buffer A; lane 5: fraction #19 collected during washing of the FPLC column with deionized water; lane 6: fraction #20 collected during washing of the FPLC column with deionized water; lane 7: fraction #21 collected during washing of the FPLC column with deionized water; lane 8: fraction #53 collected during elution of the FPLC column with a pH gradient; lane 9: fraction #54 collected during elution of the FPLC column with a pH gradient; lane 10: fraction #55 collected during elution of the FPLC column with a pH gradient.

### DETAILED DESCRIPTION OF THE INVENTION

The present invention provides a method for purifying IgY(Fc) and IgY(ΔFc) isoforms of IgY antibodies. Preferably, the source of IgY antibodies is an egg yolk obtained from an avian host. However, the method can also be used to purify IgY antibodies from the serum of an avian host.

Preferably, the avian host from which eggs or serum are collated to isolate IgY antibodies is injected, infected or immunized with an etiological agent. In one embodiment, the etiological agent can be a pathogenic or non-pathogenic organism. In another embodiment, the etiological agent can be an antigen that induces the production of desired IgY antibodies, such as naturally purified antigens, recombinant antigens, peptide-synthetic antigens, and plasmid DNA.

In one embodiment, the avian host from which eggs and serum are collated as a source of IgY antibodies is immunized with a naturally occurring, recombinant or attenuated strain of a pathological agent. In another embodiment, the avian host is spontaneously infected by a naturally occurring strain of a pathological agent. Accordingly, eggs and sera obtained from the infected avian hosts comprise polyclonal IgY antibodies against the pathological agent. Preferably, eggs and sera obtained from infected avian hosts comprise a high neutralization titer of IgY antibodies. Preferably, the neutralization titer of the IgY antibodies present in the eggs or sera is at least about 1:200, and more preferably at least 1:20,000. The term “neutralization titer” as used herein means quantification of an antibody that prevents or treats an infection and subsequent detrimental effects of the infection.

Preferably, eggs and serum are collected from those avians that belong to the biological family Anatidae, or commonly known as waterfowl. For example, the current highly pathogenic avian influenza (HPAI), H5N1, exhibits very high mortality, approaching or at 100%, to chickens and turkeys. In contrast, waterfowl birds are recognized as potential carriers of the H5N1 strain. Recent studies have shown various H5N1 strain variants cause substantially reduced or no mortality in domestic waterfowl relative that observed in chickens. The resistance of waterfowl is predicted to be due to the immunological system, and specifically antibodies, of the genus. Accordingly, eggs and sera obtained from avians of the family Anatidae are preferred as a source of IgY antibodies. Although in a preferred embodiment the avian host belongs to the family of Anatidae, other avian hosts such as chicken or turkey can also be used as a source of IgY antibodies. The most preferred avian hosts are goose, duck, turkey, ostrich or chicken.

The avian host from which IgY antibodies are obtained may be immunized or spontaneously infected with a variety of microbial pathogens (both live and heat killed), proteins (including antigen, recombinant, subunit and peptide), DNA constructs, RNA constructs, venoms, and toxins including, but not limited to, Hepatitis A virus, Hepatitis B virus, Hepatitis C virus, Human Immunodeficiency Virus, Respiratory Syncytial Virus, Cytomegalo Virus, Herpes Simplex Virus, Ectocarpus Siliculosus Virus, Vesicular Stomatital Virus, viral encephalitides (such as Eastern equine encephalomyelitis virus, Venezuelan equine encephalomyelitis virus, and Western equine encephalomyelitis virus), viral hemorrhagic fevers (such as Ebola, Marburg, Junin, Argentine, and Lassa), influenza viruses, and avian influenza viruses (sometimes called bird flu), Pasteurella multocida, Arizona Bark Scorpion venom, botulinum toxin, Plasmodium falciparum, and for use as disease biomarkers. In an embodiment, the avian host is immunized or spontaneously infected with West Nile Virus, Dengue virus, influenza virus or rabies virus. In another embodiment, the avian host is immunized with Andes-M (1.1) virus, Andes-M (optimized) virus, Guanarito virus, Junin virus, Machupo virus and Sabia virus

If sera collected from avian hosts are used as a source of IgY antibodies, the sera can be used in its natural form or may be further processed or treated. For example, the sera are preferably treated to substantially remove active forms of the transmittable disease that may be present therein.

According to the invention, the two isoforms of IgY antibodies are separated by differential precipitation of antibodies using a suitable precipitating agent. The separated fractions can be further purified, preferably using chromatographic procedures.

In one aspect of the invention, egg yolk is separated from egg white. The yolk sac is punctured and yolk contents are diluted with a solvent, such as water, preferably from about 5 to 20 parts by volume, more preferably about 9 to 15 parts by volume, per 1 part of the egg yolk. The diluted yolk suspension is preferably stirred, and the pH of the suspension is preferably adjusted to about 4.5-6.0. The acidified yolk suspension is then preferably cooled to less than 10° C. or frozen. The yolk suspension is thawed and preferably warmed if frozen, and centrifuged to obtain antibody-containing supernatant. The antibody-containing supernatant is filtered to remove lipids and bacterial contaminants. The antibody-containing supernatant is then subjected to differential precipitation in order to separate IgY(Fc) and IgY(ΔFc).

The term “precipitation” as used herein refers to a process of adding one or more non-denaturing precipitating agents to a solution in order to precipitate proteins present in the solution. The term “differential precipitation” as used herein refers to a process of separating two isoforms of IgY antibodies by adding one or more non-denaturing precipitating agents to IgY containing solution.

### Differential Precipitation of IgY Antibodies to Separate IgY(Fc) and IgY(ΔFc)

In one aspect of the invention, two isoforms of IgY antibodies are separated by differential precipitation as described below.

First precipitation: The antibody-containing supernatant is contacted with a suitable precipitating agent at a first concentration in order to obtain a first precipitate containing a population of IgY antibodies.

Second precipitation: The precipitate obtained in the first precipitation step is preferably suspended in a suitable buffer. The suspension containing the population of IgY antibodies is contacted with the same or different precipitating agent at a second concentration to obtain a second precipitate containing a majority of one IgY isoform, wherein the second concentration of the precipitating agent is less than the first concentration of the precipitating agent.

Third precipitation: The supernatant obtained in the second precipitation is contacted with the same or different precipitating agent at a third concentration to obtain a third precipitate containing a majority of the other IgY isoform, wherein the third concentration of the precipitating agent is more than the second concentration of the precipitating agent.

In one aspect of the invention, the precipitate obtained after the first precipitation step contains at least 85% of total IgY antibodies present in the original sample. Preferably, the precipitate obtained after the second precipitation step contains at least 70% (preferably at least 80% and most preferably at least 90%) of one of the two isoforms (IgY(Fc) or IgY(ΔFc)) and the precipitate obtained after the third precipitation step contains at least 70% (preferably at least 80% and most preferably at least 90%) of the other isoform. Percentages are based on the total IgY content of the precipitate.

Examples of suitable precipitating agents according to the invention include, but are not limited to caprylic acid, ammonium sulfate, sodium sulfate, magnesium sulfate, sodium chloride, potassium chloride, calcium chloride, ethanol, methanol, and polyethylene glycol.

According to the invention, the first concentration of the precipitating agent may range from about 30% to about 70%, preferably from about 35% to about 65%, and most preferably from about 40% to about 60% based on the total volume of the mixture.

According to the invention, the second concentration of the precipitating agent may range from about 15% to about 55%, preferably from about 20% to about 50%, and most preferably from about 25% to about 45% based on the total volume of the mixture.

According to the invention, the third concentration of the precipitating agent may range from about 30% to about 70%, preferably from about 35% to about 65%, most preferably from about 40% to about 60% based on the total volume of the mixture.

### Chromatographic Purification of Differentially Precipitated IgY Isoforms

The precipitated IgY(Fc) and IgY(ΔFc) antibodies may be further purified using chromatographic techniques including, but not limited to, hydrophobic chromatography, hydrophobic charge induced chromatography, adsorption chromatography, ion-exchange chromatography and affinity chromatography.

The IgY(Fc) and/or IgY(ΔFc) antibodies obtained by differential precipitation are suspended in a suitable buffer (loading buffer) and loaded onto a column. Preferably, the pH of the loading buffer used is from about 6.5 to 8.5, preferably about 7.0. Suitable loading buffers include, but are not limited to, 50 mM TrisCl (pH 8.0), 1×PBS, PBS, and 15 mM MES.

The column is washed several times and eluted with an appropriate buffer. The eluted fractions are collected, the absorbance of the eluted fractions is measured at 280 nm and the fractions are analyzed by a 4-15% gradient SDS-PAGE with coomassie staining.

The present invention is illustrated by the following examples, which are set forth to illustrate certain embodiments of the present invention and are not to be construed as limiting.

### Example 1

### A. Immunization of Geese with Dengue Virus to Induce Antigen-Specific Antibody Production

Ten geese are injected subcutaneously with killed Dengue virus (Dengue Type 2 Antigen, Microbix Biosystems Inc., Mississauga, ON, Canada, Catalog No. EL-22-02-001.0) mixed with Freund's incomplete adjuvant in 1:1 ratio. Each goose receives two subcutaneous injections containing a total of 60 μg of virus. Five geese receive two subcutaneous injections/goose of PBS mixed with Freund's incomplete adjuvant in 1:1 ratio as a control. Eggs are collected soon after laying, labeled, and stored at 4° C. until the processing for antibody purification begins.

### B. Immunization of Geese with DNA Constructs to Induce Antigen-Specific Antibody Production

A total of 24 geese are injected with 6 DNA constructs encoding for Andes-M (1.1) virus, Andes-M (optimized) virus, Guanarito virus, Junin virus, Machupo virus and Sabia virus—with 4 geese per DNA construct. Geese receiving the same DNA construct are housed together. Blood is drawn from each goose prior to immunization as a control (pre-bleed). After pre-bleed, each goose is injected intramuscularly with a total of 1 mg of an appropriate DNA construct. Eggs are collected soon after laying, labeled, and stored at 4° C. until the processing for antibody purification begins.

### Example 2

### Processing of Egg Yolk to Obtain Antibody-Containing Yolk Suspension

Eggs laid by the immunized geese were collected. After gently making an opening in the egg shell, egg white was removed and egg yolk was rinsed with several volumes of ice cold deionized water. The washed egg yolk was then transferred to an absorbent paper towel to remove residual egg white. The yolk sac was punctured and the yolk contents were transferred into a pre-weighed 1 L beaker. The yolk contents were weighed and 9× the yolk weight of ice cold deionized water was added to the beaker. The diluted yolk was gently stirred at 250-300 rpm for 15 minutes at room temperature. The yolk suspension was then acidified by adding 1 N HCl until pH 5.0. After acidification, yolk suspension was further mixed for 5 to 10 minutes and frozen at around −20° C.

### Example 3

### Differential Precipitation Using Ammonium Sulfate to Separate IgY(Fc) and IgY(ΔFc)

The frozen yolk suspension was thawed, centrifuged and filtered. The filtered IgY containing supernatant was precipitated using a saturated (100%) solution of ammonium sulfate to obtain a final ammonium sulfate concentration of about 50%. The precipitated suspension was centrifuged at 10,000×g for 15 minutes at 20° C. to obtain a precipitate containing the whole population of IgY antibodies. The precipitate was suspended in a buffer containing 50 mM TrisCl (pH 8.0) and 0.5 M arginine.

For the second ammonium sulfate precipitation, the suspended precipitate was diluted 10 times using the buffer containing 50 mM TrisCl (pH 8.0) and 0.5 M arginine and precipitated using a saturated solution of ammonium sulfate to obtain a final ammonium sulfate concentration of less than 50%. The precipitated solution was stirred and centrifuged to obtain a precipitate containing IgY(Fc) antibodies and supernatant. The precipitate was suspended in the buffer containing 50 mM TrisCl (pH 8.0) and 0.5 M arginine.

The supernatant obtained in the second ammonium sulfate precipitation was further precipitated using a saturated solution of ammonium sulfate to obtain a final ammonium sulfate concentration of more than that used for the second ammonium sulfate precipitation. The precipitated solution was stirred and centrifuged to obtain a precipitate containing IgY(ΔFc) antibodies. The precipitate was suspended in the buffer containing 50 mM TrisCl (pH 8.0) and 0.5 M arginine.

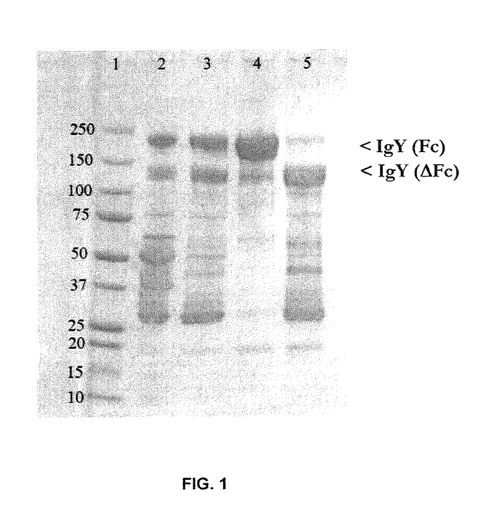

A SDS-PAGE analysis of yolk suspension and the precipitates obtained after every step of ammonium sulfate precipitation was performed (FIG. 1).

### Example 4

### Chromatographic Purification of IgY Antibodies

IgY(Fc) and IgY(ΔFc) antibodies obtained in Example 3 were further purified using various chromatographic techniques such as an ion exchange chromatography, hydrophobic chromatography or hydrophobic charge induced chromatography. For this, a suspension of precipitated antibodies was diluted 2 to 5 times with a loading buffer. For a cation exchange chromatography, hydrophobic chromatography or hydrophobic charge induced chromatography, 50 mM Tris Cl (pH 8.0) was used as a loading buffer and for an anion exchange chromatography, 50 mM sodium phosphate (pH 7.4) was used as a loading buffer. The diluted suspension was loaded onto a column using a 50 to 150 ml Superloop and injected onto the column at 2.5 to 5.0 ml/min. After washing the column with 5 to 10 column volumes of loading buffer, IgY bound to the column was washed with 2 to 5 column volumes of nonionic detergent for anion and cation exchange columns and with deionized water followed by a loading buffer containing 25 mM octanoic acid for hydrophobic charge induced chromatographic columns.

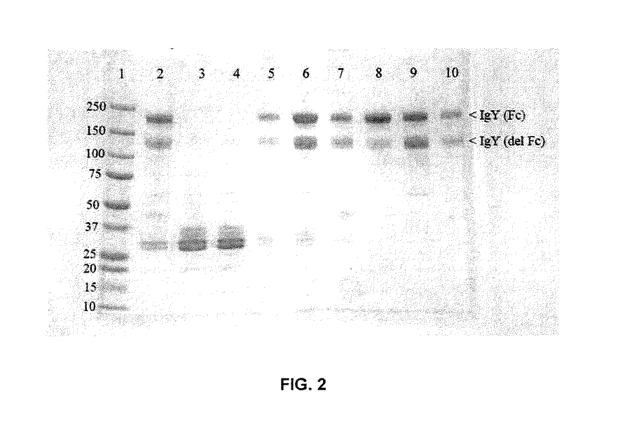

Antibodies bound to the column were eluted in 5 ml fractions using a salt gradient (0 to 1 M NaCl) for cation and anion exchange columns, a reverse salt gradient for hydrophobic columns and a pH gradient for hydrophobic charge induced chromatography. The absorbance of collected fractions was determined at 280 nm and relevant fractions were analyzed by a 4-15% gradient SDS-PAGE with coomassie staining. A representative coomassie stained SDS-PAGE gel of fractions obtained during hydrophobic charge induced chromatography of IgY(Fc) antibodies obtained during the second ammonium sulfate precipitation is shown in FIG. 2.

We claim:

1. A method for purifying IgY antibodies, comprising:
a. precipitating IgY antibodies from a sample by contacting the sample with a precipitating agent; and
b. differentially precipitating the IgY antibodies obtained in step (a) using the same or different precipitating agent to separate IgY(Fc) and IgY(ΔFc) antibodies.

2. The method according to claim 1, wherein in step (b), the IgY(Fc) antibodies are precipitated first.

3. The method according to claim 2, wherein the precipitated IgY(Fc) antibodies are physically isolated from the IgY(ΔFc) antibodies.

4. The method according to claim 1, wherein in step (b), the IgY(ΔFc) antibodies are precipitated after the precipitation of the IgY(Fc) antibodies.

5. The method according to claim 1, wherein in step (b), the IgY(ΔFc) antibodies are precipitated first.

6. The method according to claim 5, wherein the precipitated IgY(ΔFc) antibodies are physically isolated from the IgY(Fc) antibodies.

7. The method according to claim 1, wherein in step (b), the IgY(Fc) antibodies are precipitated after the precipitation of the IgY(ΔFc) antibodies.

8. The method according to claim 1, further comprising a step of chromatographically purifying one or both of the IgY(Fc) and IgY(ΔFc) antibodies obtained in step (b).

9. The method according to claim 1, wherein the IgY antibodies are obtained from egg yolk or serum of an avian host.

10. The method according to claim 9, wherein the avian host is goose, duck or chicken.

11. The method according to claim 1, wherein the IgY antibodies are obtained from goose.

12. The method according to claim 11, wherein the IgY antibodies are obtained from goose eggs.

13. The method according to claim 1, wherein the precipitating agent is independently selected from the group consisting of ammonium sulfate, sodium sulfate, magnesium sulfate, caprylic acid, ethanol, methanol, and polyethylene glycol.

14. The method according to claim 8, wherein the step of chromatographic purification comprises adsorbing the IgY antibodies to a ligand and eluting the adsorbed IgY antibodies using a pH gradient.

15. A method for purifying IgY antibodies, comprising:
a. contacting a suspension containing IgY antibodies with a precipitating agent at a first concentration to obtain a first precipitate containing the IgY antibodies;
b. contacting the precipitate containing the IgY antibodies obtained in step (a) with the same or different precipitating agent at a second concentration to obtain a second precipitate containing a majority of one IgY isoform and a supernatant containing a majority of the other IgY isoform, wherein the second concentration is less than the first concentration;
c. contacting the supernatant obtained in step (b) with the same or different precipitating agent at a third concentration to obtain a third precipitate containing a majority of the other IgY isoform, wherein the third concentration is more than the second concentration; and
d. purifying the antibodies obtained in steps (b) and/or (c).

16. The method according to claim 16, wherein in step (d), the antibodies obtained in steps (b) and/or (c) are purified using hydrophobic charge induction chromatography.

17. An isolated IgY(Fc) antibody obtained from goose eggs by the method of claim 1.

18. An isolated IgY(ΔFc) antibody obtained from goose eggs by the method of claim 1.

19. A composition comprising IgY antibodies against a transmittable viral disease, wherein the IgY antibodies are obtained from goose eggs by the method of claim 1.
